SEQUENCE LISTING (40 sequences)

SEQ ID NO: 1 - DNA, 709 bp, synthetic construct
  source 1..709: mol_type = other DNA
  misc_feature 1..709: note = MND-HTLV-1 Promoter Sequence
gatatctgcagaattcgcccttatggggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 2 - DNA, 817 bp, synthetic construct
  source 1..817: mol_type = other DNA
  misc_feature 1..817: note = MND-HTLV-2 Promoter Sequence
gatatcgagatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatcgatggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 3 - DNA, 798 bp, synthetic construct
  source 1..798: mol_type = other DNA
  misc_feature 1..798: note = Anti-CD19 scFv
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 4 - DNA, 921 bp, synthetic construct
  source 1..921: mol_type = other DNA
  misc_feature 1..921: note = Anti-CD33 scFv
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 5 - DNA, 1044 bp, synthetic construct
  source 1..1044: mol_type = other DNA
  misc_feature 1..1044: note = Anti-CD123 scFv
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 6 - DNA, 252 bp, Homo sapiens
  source 1..252: mol_type = genomic DNA
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 7 - DNA, 72 bp, Homo sapiens
  source 1..72: mol_type = genomic DNA
ccattttttttctgctgcttcatcgctgtagccatgggaatccgtttcattattatggtaacaatatggagt

SEQ ID NO: 8 - DNA, 252 bp, Homo sapiens
  source 1..252: mol_type = genomic DNA
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 9 - DNA, 73 bp, Homo sapiens
  source 1..73: mol_type = genomic DNA
gtctctttctgcttggtgatggtactcctttttgcagtggacacaggactatatttctctgtgaagacaaaca

SEQ ID NO: 10 - DNA, 252 bp, Homo sapiens
  source 1..252: mol_type = genomic DNA
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 11 - DNA, 72 bp, Homo sapiens
  source 1..72: mol_type = genomic DNA
ctggtgcctgtgttctgtggactcctcgtagccaagagcctggtgctgtcagccctgctcgtctggtggggg

SEQ ID NO: 12 - DNA, 252 bp, Homo sapiens
  source 1..252: mol_type = genomic DNA
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 13 - DNA, 72 bp, Homo sapiens
  source 1..72: mol_type = genomic DNA
gctggtacagtcctcctccttcgggctggattctatgctgtcagctttctctctgtggccgtgggcagcacc

SEQ ID NO: 14 - DNA, 252 bp, Homo sapiens
  source 1..252: mol_type = genomic DNA
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 15 - DNA, 72 bp, Homo sapiens
  source 1..72: mol_type = genomic DNA
atgggcctggcctttctagtcctggtggctctagtgtggttcctggttgaagactggctcagcaggaagagg

SEQ ID NO: 16 - DNA, 252 bp, Homo sapiens
  source 1..252: mol_type = genomic DNA
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 17 - DNA, 69 bp, Homo sapiens
  source 1..69: mol_type = genomic DNA
gttctgattgggacctcagtggtcaaaatccctttcaccatcctcctcttctttctccttcatcgctgg

SEQ ID NO: 18 - DNA, 210 bp, Homo sapiens
  source 1..210: mol_type = genomic DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 19 - DNA, 78 bp, Homo sapiens
  source 1..78: mol_type = genomic DNA
ctcactgccgaggtcctaggaatcatttgcattgtcctgatggccactgtgttaaaaacaatagttcttattcctttc

SEQ ID NO: 20 - DNA, 252 bp, Homo sapiens
  source 1..252: mol_type = genomic DNA
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 21 - DNA, 72 bp, Homo sapiens
  source 1..72: mol_type = genomic DNA
atctacatctgggcgcccttggccgggacttgtggggtccttctcctgtcactggttatcaccctttactgc

SEQ ID NO: 22 - DNA, 252 bp, Homo sapiens
  source 1..252: mol_type = genomic DNA
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 23 - DNA, 84 bp, Homo sapiens
  source 1..84: mol_type = genomic DNA
attccgtggctcggccacctcctcgtgggcctcagcggggcttttggcttcatcatcttagtgtacttgctgatcaactgcagg

SEQ ID NO: 24 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 25 - DNA, 182 bp, Homo sapiens
  source 1..182: mol_type = genomic DNA
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 26 - DNA, 512 bp, synthetic construct
  source 1..512: mol_type = other DNA
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 27 - DNA, 251 bp, Homo sapiens
  source 1..251: mol_type = genomic DNA
agaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtg

SEQ ID NO: 28 - DNA, 242 bp, Homo sapiens
  source 1..242: mol_type = genomic DNA
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 29 - DNA, 167 bp, Homo sapiens
  source 1..167: mol_type = genomic DNA
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 30 - DNA, 351 bp, Homo sapiens
  source 1..351: mol_type = genomic DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 31 - DNA, 210 bp, Homo sapiens
  source 1..210: mol_type = genomic DNA
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 32 - DNA, 144 bp, Homo sapiens
  source 1..144: mol_type = genomic DNA
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 33 - DNA, 141 bp, Homo sapiens
  source 1..141: mol_type = genomic DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 34 - DNA, 144 bp, Homo sapiens
  source 1..144: mol_type = genomic DNA
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 35 - DNA, 861 bp, Homo sapiens
  source 1..861: mol_type = genomic DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 36 - DNA, 573 bp, Mus musculus
  source 1..573: mol_type = genomic DNA
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 37 - DNA, 279 bp, Homo sapiens
  source 1..279: mol_type = genomic DNA
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 38 - DNA, 660 bp, Homo sapiens
  source 1..660: mol_type = genomic DNA
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 39 - DNA, 858 bp, Homo sapiens
  source 1..858: mol_type = genomic DNA
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 40 - DNA, 1542 bp, Homo sapiens
  source 1..1542: mol_type = genomic DNA
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

(19) United States
(12) Patent Application Publication
(10) Pub. No.: US 2023/0330228 A1
(43) Pub. Date: Oct. 19, 2023

(54) HYBRID PROMOTERS, VECTORS CONTAINING SAME AND METHODS OF USE

(71) Applicant: Wugen, Inc., Saint Louis, MO (US)

(72) Inventors: Nathan POMPER, St. Louis, MO (US); John DEAN, Clayton, MO (US); Elizabeth SCHRAMM, O'Fallon, IL (US); Matthew COOPER, St. Louis, MO (US); Ryan SULLIVAN, Ballwin, MO (US)

(21) Appl. No.: 18/188,410

(22) Filed: Mar. 22, 2023

Related U.S. Application Data
(60) Provisional application No. 63/322,384, filed on Mar. 22, 2022.

(51) Int. Cl. A61K 39/00; C12N 15/86; C07K 14/705; C07K 14/725; C07K 14/735; C07K 14/715; A61P 35/00

(52) U.S. Cl. CPC A61K 39/4613; C12N 15/86; C07K 14/70503; C07K 14/7051; C07K 14/70535; C07K 14/7155; A61K 39/464499; A61K 39/4631; A61P 35/00; C12N 2740/15043; C07K 2319/033; C07K 2319/03; C07K 2319/33; A61K 2039/5156; C07K 2317/732; A61K 2239/21; A61K 2239/22

(57) ABSTRACT

The present disclosure provides hybrid promoter sequences comprising an MND promoter and HTLV enhancer capable of driving high levels of sustained expression of a heterologous sequence in immune cells, particularly Natural Killer (NK) cells. The disclosure also provides compositions comprising such vectors, immune cells which have been genetically modified to contain the vectors, as well as methods of using the same for inducing immune responses and treating cancer and other conditions.

### CROSS-REFERENCE TO RELATED APPLICATIONS

This application claims the benefit of U.S. Provisional Application No. 63/322,384, filed Mar. 22, 2022, the contents of all of which is herein incorporated by reference in their entirety.

### STATEMENT REGARDING SEQUENCE LISTING

The Sequence Listing XML associated with this application is provided in XML file format and is hereby incorporated by reference into the specification. The name of the XML file containing the Sequence Listing XML is WUGE_001_01US_ST26.xml. The XML file is 51,701 bytes, and created on Mar. 17, 2023, and is being submitted electronically via USPTO Patent Center.

### TECHNICAL FIELD

The present invention relates to hybrid promoter sequences capable of driving high levels of sustained expression of a heterologous sequence in immune cells, particularly Natural Killer (NK) cells. The invention also relates to compositions comprising such vectors, immune cells which have been genetically modified to contain the vectors, as well as methods of using the same for inducing immune responses and treating cancer and other conditions.

### BACKGROUND

Exogenous gene expression in cellular therapeutics and scientific research requires the use of transcriptional regulatory sequences (e.g., promoters) to dictate the timing, extent and cellular specificity of gene expression. A wide variety of well-known promoters have been used across a multitude of cellular systems. These include promoters derived from many different sources, including endogenous human genes, animal genes, bacterial genes, viral genes and synthetic sequences. However, generally speaking, most commonly used promoters available for cellular therapeutics and other applications exhibit either weak or silenced activity in certain immune cells of interest, particularly Natural Killer (NK) cells, which limits or prevents their utility in therapeutic applications involving NK cells. As such, there is an important and unmet need for identifying transcriptional regulatory sequences capable of providing high levels of expression and sustained expression of heterologous sequences in immune cells, including NK cells. The present invention meets these needs and offers other related advantages.

### SUMMARY

According to one aspect of the present disclosure, there is provided a nucleic acid vector comprising a hybrid promoter sequence operably linked to a heterologous sequence, where the hybrid promoter sequence has activity in immune cells, and where the hybrid promoter sequence comprises: (i) an MND promoter sequence and (ii) an HTLV enhancer sequence.

In a more particular embodiment of the disclosure, there is provided a nucleic acid vector comprising a hybrid promoter sequence operably linked to a heterologous sequence, where the hybrid promoter sequence has activity in immune cells, and where the hybrid promoter sequence comprises: (i) an MND promoter sequence and (ii) an HTLV enhancer sequence, where the hybrid promoter sequence comprises the sequence of SEQ ID NO: 1 or SEQ ID NO: 2 or a fragment or variant thereof having at least 90% identity thereto.

In a more particular embodiment of the disclosure, there is provided a nucleic acid vector comprising a hybrid promoter sequence operably linked to a heterologous sequence, where the hybrid promoter sequence has activity in immune cells, and where the hybrid promoter sequence comprises: (i) an MND promoter sequence and (ii) an HTLV enhancer sequence, where the MND promoter sequence comprises residues 1-357 of SEQ ID NO: 1 or residues 1-548 of SEQ ID NO: 2.

In a more particular embodiment of the disclosure, there is provided a nucleic acid vector comprising a hybrid promoter sequence operably linked to a heterologous sequence, where the hybrid promoter sequence has activity in immune cells, and where the hybrid promoter sequence comprises: (i) an MND promoter sequence and (ii) an HTLV enhancer sequence, where the HTLV enhancer sequence comprises residues 441-709 of SEQ ID NO: 1 or residues 549-817 of SEQ ID NO: 2.

In some embodiments, the immune cell in which the hybrid promoter has activity is an immune effector cell. In some embodiments, the immune cell in which the hybrid promoter has activity is an NK cell. In some embodiments, the immune cell in which the hybrid promoter has activity is a memory-like (ML) NK cell. In some embodiments, the immune cell in which the hybrid promoter has activity is a T cell. In some embodiments, the immune cell in which the hybrid promoter has activity is a B cell.

In some embodiments, the heterologous sequence operably linked to an MND-HTLV promoter sequence is a nucleic acid sequence encoding a chimeric antigen receptor. In a more particular embodiment, the heterologous sequence operably linked to an MND-HTLV promoter sequence is a nucleic acid sequence encoding a chimeric antigen receptor, where the chimeric antigen receptor comprises an extracellular domain that binds a target antigen, a transmembrane domain and one or more intracellular signaling domains.

In another particular embodiment, the heterologous sequence operably linked to an MND-HTLV promoter sequence is a nucleic acid sequence encoding a chimeric receptor, where the chimeric receptor comprises an extracellular domain chosen or derived from an Fc receptor, a transmembrane domain and one or more intracellular signaling domains.

In some embodiments, the promoters of the present disclosure are used in the context of targeted cellular micropharmacies (e.g., Cancers (Basel). 2020 August; 12(8): 2175) for expressing/delivering therapeutic molecules of interest in a targeted fashion. For example, in some embodiments, the heterologous sequence operably linked to an MND-HTLV promoter sequence is a nucleic acid sequence encoding a cytokine, chemokine, an immune activating moiety, an antibody, an enzyme, an anti-tumor molecule and/or another therapeutic molecule of interest. In a more particular embodiment, the heterologous sequence operably linked to an MND-HTLV promoter sequence is a nucleic acid sequence encoding a cytokine, such as a cytokine selected from the list of IL-2, IL-4, IL-15, IL-12, IL and others. In another particular embodiment, the heterologous sequence operably linked to an MND-HTLV promoter sequence is a nucleic acid sequence encoding a chemokine, where the chemokine is selected, for example, from CXCL1, CXCL2, CCL4, CCL5, XCL1 and others. In another particular embodiment, the heterologous sequence operably linked to an MND-HTLV promoter sequence is a nucleic acid sequence encoding an antagonist of immunosuppressive cytokines, such as an scFv or antagonistic protein, where the target is selected, for example, from TGFb, PGE2, and the like.

In some embodiments, the heterologous sequence operably linked to an MND-HTLV promoter sequence is a nucleic acid sequence encoding a chimeric antigen receptor, where the chimeric antigen receptor comprises an extracellular domain that binds a target selected from the group consisting of mesothelin, CD2, CD3, CD4, CD5, CD7, BAFF-R, gp120, gp41, BCMA, CD123, CD138, CD19, CD20, CD22, CD33, CD38, CD5, IgK, LeY, NKG2D-Ligands, MICA, MICE, ULBP1, ULBP2, ULBP3, ULBP4, ULBP5, ULBP6, ROR1, WT1, c-MET, CAIX, CD133, CD171, CD70, CEA, EGFR, EGFRvIII, EPCAM, EphA2, FAP, GD2, GPC3, Her2, HPV16-E6, IL13Ra2, LeY, MAGEA3, MAGEA4, MART1, MSLN, MUC1, MUC16, NY-ESO-1, PD-L1, PSCA, PSMA, ROR1, VEGFR2, BAFF-R and SLAM-F7.

In some embodiments, the heterologous sequence operably linked to an MND-HTLV promoter sequence is a nucleic acid sequence encoding a chimeric antigen receptor, where the chimeric antigen receptor comprises, a transmembrane domain selected from the group consisting of NKG2D, FcγRIIIa, NKp44, NKp30, NKp46, actKIR, NKG2C, CD8a, CD28 and IL15Rb.

In some embodiments, the heterologous sequence operably linked to an MND-HTLV promoter sequence is a nucleic acid sequence encoding a chimeric antigen receptor, where the chimeric antigen receptor comprises an intracellular signaling domain selected from the group consisting of CD137/41 BB, DNAM-1, NKp80, 2B4, NTBA, CRACC, CD2, CD27, CD79a CD79b, CD132 one or more integrins, IL-15R, IL-18R, IL-12R, IL-21 R, IRE1a, and combinations thereof.

In some embodiments, a vector according to the present disclosure comprising an MND-HTLV promoter is a viral vector. In a more particular embodiment, the viral vector is a lentiviral vector.

In some embodiments, a vector according to the present disclosure comprising an MND-HTLV promoter is a viral vector, including but not limited to Lentiviral vector, Retroviral vector, Adenoviral vector, Adeno-associated viral vectors or non-viral vectors including but not limited to transposons such as sleeping beauty.

According to another aspect of the present disclosure there is provided a genetically modified immune cell comprising a vector as disclosed herein. In some embodiments, the genetically modified immune cell is an NK cell or ML NK cell. In some embodiments, the genetically modified immune cell is a B cell or a T cell. In some embodiments, the modified immune cell is optionally deficient for NKG2A, CD8, EP2, EP4, SIPRP alpha or CISH expression, activity or signaling.

According to a more particular aspect of the present disclosure, there is provided a genetically modified ML NK cell comprising a chimeric antigen receptor where the chimeric antigen receptor is expressed under the control of a hybrid promoter sequence comprising: (i) an MND promoter sequence and (ii) an HTLV enhancer sequence, wherein the cell is optionally deficient for NKG2A, CD8, EP2, EP4, SIPRP alphaor CISH expression, activity or signaling. In some embodiments of this aspect of the disclosure, the hybrid promoter sequence comprises the sequence of SEQ ID NO: 1 or SEQ ID NO: 2 or a fragment or variant thereof having at least 90% identity thereto. In other embodiments, the MND promoter sequence comprises residues 1-357 of SEQ ID NO: 1 or residues 1-548 of SEQ ID NO: 2. In other embodiments, the HTLV enhancer sequence comprises residues 441-709 of SEQ ID NO: 1 or residues 549-817 of SEQ ID NO: 2.

According to another aspect of the present disclosure, there is provided a method of inducing an immune response to a disease in a subject in need thereof comprising administering to the subject a genetically modified immune cell as described herein.

According to another aspect of the present disclosure, there is provided a method of treating cancer in a subject in need thereof comprising administering to the subject a genetically modified immune cell as described herein, wherein the target antigen is a cancer-associated target antigen.

### BRIEF DESCRIPTION OF THE DRAWINGS

FIG. 1 shows the sequence of an illustrative MND-HTLV promoter sequence referred to as MND-HTLV-1 (SEQ ID NO: 1).

FIG. 2 shows the sequence of an illustrative MND-HTLV promoter sequence referred to as MND-HTLV-2 (SEQ ID NO: 2).

FIG. 3 compares the GFP expression levels for a number of different promoters observed over time in NK cells following lentivirus transduction. These results demonstrate that an MND-HTLV promoter has superior promoter activity in NK cells relative to every other promoter tested.

FIG. 4A compares CD34 expression levels by mean fluorescence intensity (MFI) driven by the MND-HTLV1 promoter and driven by the MND promoter.

FIG. 4B shows cytotoxicity of different promoter driven receptor expression NK cells against SKOV3 tumor cells in conjunction with trastuzumab (tras). The data demonstrates that NK cells transduced with the MND-HTLV1 driven receptor led to increased cytotoxicity of tumor cells.

### DETAILED DESCRIPTION

The following definitions and descriptions are provided to better understand the present invention and to guide those of ordinary skill in the art in the practice of the present invention. Unless otherwise noted, terms are to be understood according to conventional usage by those of ordinary skill in the relevant art.

A “promoter” is generally understood as a nucleic acid sequence that is recognized by an RNA polymerase which binds to the promoter and directs transcription of a nucleic acid sequence operably linked to the promoter. A promoter can include necessary nucleic acid sequences near the start site of transcription, such as, in the case of a polymerase II type promoter, a TATA element. A promoter can also optionally include enhancer or repressor elements. An inducible promoter is generally understood as a promoter that mediates transcription of an operably linked gene in response to a particular stimulus.

The term “enhancer” refers to a nucleic acid sequence which contains sequences capable of providing enhanced transcription and in some instances can function independent of their orientation relative to another control sequence. An enhancer can function cooperatively or additively with promoters and/or other enhancer elements.

The terms “heterologous gene” or “heterologous nucleic acid” or “heterologous sequence”, as used herein, refer to a sequence that originates from a source foreign to the particular host cell or, if from the same source, is modified from its original form. Thus, a heterologous nucleic acid in a host cell can include sequences that are endogenous to the particular host cell but where the sequences have been modified from their wild type forms. A heterologous sequence can also include a sequence that is endogenous to the particular host cell but is under the control of a promoter sequence that is not naturally associated with the sequence. The terms also include non-naturally occurring multiple copies of a naturally occurring DNA sequence.

“Operably linked” or “functionally linked” refers to the association of nucleic acid sequences on a single nucleic acid fragment so that the function of one is affected by the other in an intended manner. For example, a regulatory DNA sequence (such as a promoter) is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

A “transcribable nucleic acid molecule” as used herein refers to any nucleic acid molecule capable of being transcribed into an RNA molecule.

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

Methods are known for introducing constructs into a cell in such a manner that the transcribable nucleic acid molecule is transcribed into a functional mRNA molecule that is translated and therefore expressed as a protein product.

Constructs may also be constructed to be capable of expressing antisense RNA molecules, to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999).

The term “untransformed” refers to normal cells that have not been through the transformation process.

As used herein, the term “genetically engineered” or “genetically modified” refers to the addition of extra genetic material in the form of DNA or RNA into the total genetic material in a cell. The terms, “genetically modified cells,” “modified cells,” and “redirected cells” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide.

“Wild-type” refers to a virus or organism found in nature without any known mutation.

Design, generation, and testing of the variant nucleotides, within transcriptional regulatory sequences (e.g., promoters) as well as encoded polypeptides, having the herein required percent identities and retaining a required promoter activity or activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 1 19-123; Ghadessy et al. (2001) Proc Natl Acad Sci USA 98(8) 4552-4557. Thus, one skilled in the art could generate a large number of nucleotide and/or polypeptide variants having, for example, at least 90-99% identity or 95-99% identity to the reference sequence described herein and screen such for desired phenotypes according to methods routine in the art.

In some embodiment, an MND-HTLV promoter sequences comprises one or more nucleotide insertions, deletions, substitutions, or modifications, relative to the specific MND-HTLV promoter sequences disclosed herein, such that increased or stabilized MND-HTLV promoter activity is achieved. In some embodiments, an MND-HTLV promoter sequences comprises 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more, nucleotide insertions, deletions, substitutions, or modifications, relative to the specific MND-HTLV promoter sequences disclosed herein, such that increased or stabilized MND-HTLV promoter activity is achieved.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y*100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, mechanoporation, and the like. The transfected cells can be selected and propagated to provide recombinant host cells that comprise the expression vector stably integrated in the host cell genome.

Exemplary nucleic acids which may be introduced to a vector or host cell include, for example, exogenous sequences or sequences which originate with or are present in the same species, but are incorporated into recipient cells by genetic engineering methods. The term “exogenous” refers to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNAWhitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended.

For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

### MND-HTLV Hybrid Promoters and Vectors Containing Same

As described herein, the present disclosure relates generally to MND-HTLV promoter sequences having activity in immune cells, especially Natural Killer (NK) cells and memory-like NK (ML-NK) cells as well as vectors comprising an MND-HTLV promoter sequence operably linked to a heterologous sequence of interest, such as a heterologous sequence encoding a chimeric antigen receptor (CAR).

We have found that most of the commonly used promoters available for cellular therapeutics and other applications exhibit either weak or silenced activity in certain immune cells of interest, particularly Natural Killer (NK) cells, which limits or prevents their utility in therapeutic applications involving NK cells. We sought to identify promoter sequences that could overcome such limitations by providing robust and persistent expression of heterologous sequences in vitro and in vivo. Comparative studies employing many different promoters (some naturally occurring, some not) identified a hybrid promoter sequence that was unexpectedly superior to all the rest.

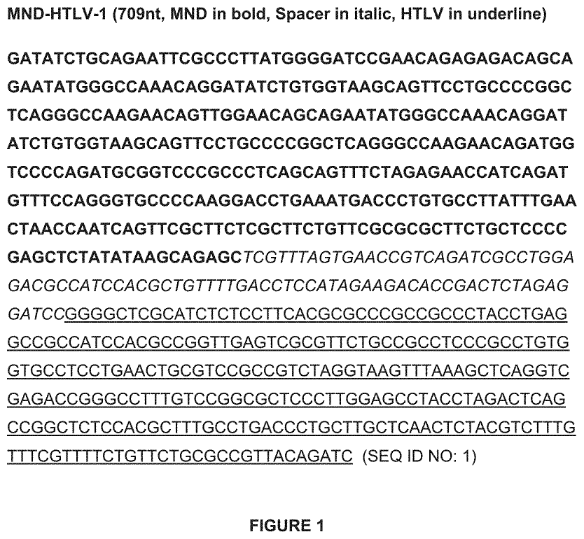

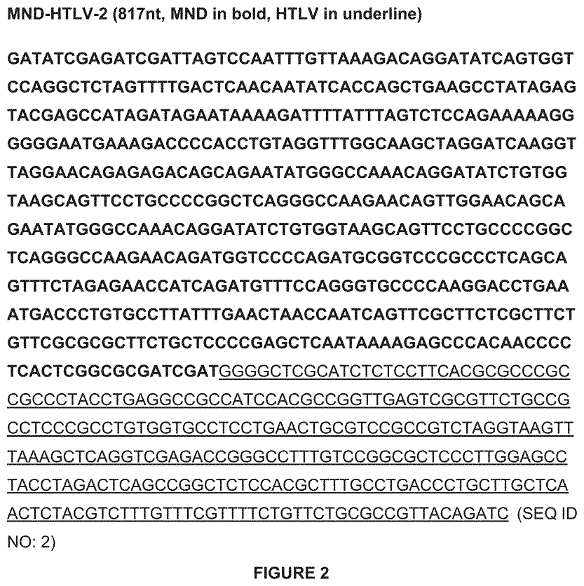

The hybrid promoter includes transcriptional regulatory sequences derived from two different sources, one being an MND promoter sequence and the other being a Human T-Lymphotropic Virus (HTLV) enhancer sequence. Sequences from these two sources were fused to generate the non-naturally occurring hybrid promoter sequences referred to as MND-HTLV-1 (SEQ ID NO: 1) and MND-HTLV-2 (SEQ ID NO: 2). Additional details about these hybrid promoters and the sequences that make them up are shown in FIGS. 1 and 2, respectively.

Therefore, in some embodiments, a hybrid MND-HTLV promoter sequence comprises the sequence of SEQ ID NO: 1 or SEQ ID NO: 2 or a fragment or variant thereof having at least 70% or 75% or 80% or 85% or 90% or 95% or 96% or 97% or 98% or 99% identity thereto, wherein the fragment or variant thereof has improved promoter activity or other features in the desired cell type of interest, such as NK cells.

In other embodiments, the MND promoter sequence that is present in the MND-HTLV promoter comprises or consists essentially of residues 1-357 of SEQ ID NO: 1 or residues 1-548 of SEQ ID NO: 2, or a fragment or variant thereof having at least 80% or 85% or 90% or 95% or 96% or 97% or 98% or 99% identity thereto, wherein the fragment or variant thereof has improved promoter activity or other features in the desired cell type of interest, such as NK cells.

In other embodiments, the HTLV enhancer sequence present within the MND-HTLV promoter comprises or consists essentially of residues 441-709 of SEQ ID NO: 1 or residues 549-817 of SEQ ID NO: 2, or a fragment or variant thereof having at least 80% or 85% or 90% or 95% or 96% or 97% or 98% or 99% identity thereto, wherein the fragment or variant thereof has improved promoter activity or other features in the desired cell type of interest, such as NK cells.

The present disclosure also provides vectors and host cells which contain or include a hybrid MND-HTLV promoter sequence operably linked to a heterologous sequence of interest. The vectors used could be of any type, illustrative examples of which include plasmids (e.g., DNA plasmids or RNA plasmids), transposons, cosmids, bacterial artificial chromosomes and viral vectors (e.g., replication defective retroviruses, lentiviruses, adenoviruses and the like).

### Genetically Modified Cells

The present disclosure also provides genetically modified cells which contain a vector comprising a hybrid MND-HTLV promoter operably linked to a heterologous sequence, as well as methods of making and using the same.

In some embodiments, a vector comprising a hybrid MND-HTLV promoter operably linked to a heterologous sequence (e.g., a CAR) is introduced and expressed in immune effector cells so as to redirect their specificity to a target antigen of interest. An “immune effector cell,” includes any cell of the immune system that has one or more effector functions (e.g., cytotoxic cell killing activity, secretion of cytokines, induction of antibody-dependent cellular cytotoxicity (ADCC) and/or complement-dependent cytotoxicity (CDC).

Immune effector cells of the invention can be autologous/autogeneic (self) or non-autologous (non-self, e.g., allogeneic, syngeneic or xenogeneic). “Autologous” refers to cells from the same subject. “Allogeneic” refers to cells of the same species that differ genetically to the cell in comparison. “Syngeneic” refers to cells of a different subject that are genetically identical to the cell in comparison. “Xenogeneic” refers to cells of a different species to the cell in comparison. In preferred embodiments, the cells of the invention are allogeneic.

Illustrative immune effector cells used with promoters and vectors disclosed herein can include essentially any immune cell in which the MND-HTLV promoter sequence has a desired level of activity. In some embodiments, the immune effector cell is a T cell, B cell, a macrophage or an NK cell. Immune effector cells can also include progenitors of effector cells wherein such progenitor cells can be induced to differentiate into an immune effector cell in vivo or in vitro.

The vectors and CAR constructs of the present disclosure are particularly engineered for enhanced activity and performance in NK cells. The term “NK cells” can refer generally to NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells.

NK Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize, and eliminate malignant cells, and are amendable to cellular engineering.

Memory, Memory-Like, and CIML NK Cells

In addition to their innate cytotoxic and immunostimulatory activity, NK cells constitute a heterogeneous and versatile cell subset, including persistent memory NK populations, in some cases also called memory-like or cytokine-induced-memory-like (CIML) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+NK cells were reduced. Finally, unlike in vitro differentiated ML NK cells, in vivo differentiated ML NK cells did not express CD25.

Cytokine-Induced Memory-Like Natural Killer Cells (CIML-NKs)

NK cells may be induced to acquire a memory-like phenotype, for example by priming (preactivation) with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8(357):357). Memory NK cells may also be identified by observed in vitro and in vivo properties, such as enhanced effector functions such as cytotoxicity, improved persistence, increased IFN-γ production, and the like, when compared to a heterogenous NK cell population.

### Chimeric Antigen Receptors

In some embodiments, an MND-HTLV promoter sequence according to the present disclosure is operably linked to a chimeric antigen receptor (CAR) to be expressed in an immune effector cell (e.g., an NK cell). The CAR sequence operably linked to an MND-HTLV promoter can be essentially any CAR type known and described in the art.

Many CARs have been described. They are generally designed in a modular fashion that comprise an extracellular target-binding domain, a hinge region, a transmembrane domain that anchors the CAR to the cell membrane, and one or more intracellular domains that transmit activation signals. Introduction of CAR molecules into a cell can successfully redirect cells with additional antigen specificity and provides the necessary signals to drive full immune cell activation. Furthermore, the CAR construct moieties can be operably linked with a linker. A linker can be any nucleotide sequence capable of linking the moieties described herein. For example, the linker can be any amino acid sequence suitable for this purpose (e.g., of a length of 9 amino acids).

In some embodiments of the present disclosure, a CAR sequence comprises at least an extracellular domain that binds a target antigen, a transmembrane domain and one or more intracellular signaling domains.

Chimeric Antigen Receptor NK (CAR NK) Cells

In certain more particular embodiments, an MND-HTLV promoter according to the present disclosure is operably linked to a CAR sequence for promoting expression of the CAR sequence in a chimeric antigen receptor NK cell (e.g., a memory-like CAR NK cell) or other cell type, as described in WO2020/097164, the contents of which are incorporated herein by reference.

a. Illustrative Extracellular Domains

Illustrative targeting antibody fragments against a disease-associated antigen can comprise single-chain variable fragments (scFvs). scFvs, as described herein can be any scFv capable of binding to a target antigen or target antigen epitope. For example, the scFvs can target an antigen associated with an infectious disease, a bacterial infection, a virus, or a cancer. scFvs can be against any antigen known in the art, such as those described in U.S. application Ser. No. 15/179,472, and is incorporated by reference in its entirety.

Targeting antibody fragments or scFvs, as described herein, can be against any tumor-associated antigen (TAA). A TAA can be any antigen known in the art to be associated with tumors.

Illustrative examples of scFvs incorporated into CARs can include those that bind, for example, to mesothelin, CD2, CD3, CD4, CD5, CD7, BAFF-R, gp120, gp41, BCMA, CD123, CD138, CD19, CD20, CD22, CD33, CD38, CD5, IgK, LeY, NKG2D-Ligands, MICA, MICB, ULBP1, ULBP2, ULBP3, ULBP4, ULBP5, ULBP6, ROR1, WT1, c-MET, CAIX, CD133, CD171, CD70, CEA, EGFR, EGFRvIII, EPCAM, EphA2, FAP, GD2, GPC3, Her2, HPV16-E6, IL13Ra2, LeY, MAGEA3, MAGEA4, MART1, MSLN, MUC1, MUC16, NY-ESO-1, PD-L1, PSCA, PSMA, ROR1, VEGFR2, BAFF-R and SLAM-F7.

The antigen-binding capability of the CAR is defined by the extracellular scFv, not the targeted antigen. The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the ML NK cell surface or whether the CARML NK cells target the antigen and signal. In addition, the length and/or composition of the variable domain linker can contribute to the stability or affinity of the scFv.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

CAR scFv affinities, modified through mutagenesis of complementary-determining regions while holding the epitope constant, or through CAR development with scFvs derived from therapeutic antibodies against the same target, but not the same epitope, can change the strength of the ML NK cell signal and allow CAR NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with CAR NK cells (as opposed to soluble antibodies), pre-clinical testing of normal tissues for expression of the target, and susceptibility to on-target toxicities, requires live-cell assays rather than immunohistochemistry on fixed tissues.

The scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example gp120 and gp41 on HIV-infected cells. scFv sequences and specificities:

Anti-CD19 scFv
(SEQ ID NO: 3)
ATGGCCCT GCCCGTGACCGCTCTCCT GCTGCCTCTGGCCCT GCTCCTCCATGCT GCCAG
ACCCGACATCCAGAT GACACAGACAACCAGCAGCCT GTCCGCTTCCCTCGGAGACAGGG
TGACAATTTCCTGCAGGGCCAGCCAGGACAT CAGCAAGTACCTGAACTGGTACCAGCAG
AAACCCGACGGCACCGT CAAGCTCCT GATCTACCACACCAGCAGACTGCACAGCGGAGT
GCCTTCCAGGTTCAGCGGCAGCGGCT CCGGCACCGATTACT CCCT GACCATTAGCAACT
TAGAACAGGAGGACATT GCCACCTACTTTTGTCAGCAGGGCAACACCCTCCCCTACACC
TTTGGAGGCGGAACCAAGTTAGAAAT
CACCGGCGGCGGCGGCAGCGGAGGAGGAGGCAG
CGGAGGCGGAGGCTCCGAGGTGAAACTGCAGGAGAGCGGCCCCGGACTGGTCGCCCCTA
GCCAATCCCTCTCCGTCACCTGCACCGTGAGCGGAGTGAGCCTGCCTGACTACGGAGTG
AGCT GGAT CAGACAGCCCCCTAGGAAAGGACTGGAATGGCT GGGCGTGATTTGGGGCAG
CGAGACCACCTATTACAACAGCGCCCTGAAGTCCAGACTGACAAT CATCAAGGACAATA
GCAAAAGCCAAGT GTTT CTGAAGATGAACAGCCTGCAGACCGATGACACCGCCATCTAT
TATT GCGCCAAGCACTACTACTACGGAGGAAGCTACGCTAT GGATTATTGGGGCCAAGG
CACAAGCGTGACCGTCAGCAGCGCGGCCGCC
Anti-CD33 scFv
(SEQ ID NO: 4)
ATGGCCTTACCAGTGACCGCCTTGCT CCTGCCGCTGGCCTT GCTGCTCCACGCCGCCAG
GCCGATGGAAAAGGATACACTGTTGTTGTGGGTTCT GCTCCTGTGGGTGCCCGGCAGCA
CCGGAGATATTGT GCTGACGCAGTCT CCTGCATCACTCGCCGTGT CTCTGGGCCAGCGC
GCTACCAT CAGCT GCAGAGCCT CTGAAAGTGTTGACAATTATGGAATTTCTTTCAT GAA
TTGGTTCCAGCAGAAGCCTGGCCAGCCCCCGAAACT CCTCATATATGCCGCGTCTAATC
AGGGCTCT GGGGT CCCT GCTAGATTTTCTGGCAGCGGCTCCGGCACCGACTTCAGTCTG
AATATACATCCCATGGAAGAAGACGATACCGCCATGTACTTTTGCCAACAATCTAAGGA
GGTGCCTT GGACGTTCGGCGGCGGTACGAAGCTGGAAATTAAGGGCGGCGGGGGAAGCG
GCGGGGGGGGATCAGGCGGGGGTGGCTCCGGAGGCGGTGGAAGTATGGGCTGGAGTTG
G ATCTTCCTTTTCCTTCTTTCTGGTACCGCGGGAGTGCACTCTGAGGTGCAGCTCCAGCA
GTCCGGCCCCGAGCTCGTCAAGCCTGGGGCCAGTGT CAAGATTTCCTGTAAGGCATCTG
GAT AT AC CT TT AC AGAT T AC AAT AT G CAT TGGGT GAAAC AGT C AC AT
GGAAAGT C ACT C GAGT GGAT
CGGATACATTTACCCTTACAATGGAGGAACCGGATATAATCAGAAGTTTAA
GAGCAAGGCCACACTCACGGTGGACAATTCTTCATCTACAGCCTACATGGATGTTCGGT
CTCT GACTTCCGAGGATAGTGCGGTGTATTACTGCGCCAGGGGACGCCCCGCTATGGAT
TACT GGGGGCAGGGAACCTCTGTAACAGTTAGCTCA
Anti-CD123 scFv
(SEQ ID NO: 5)
ATGGCCTTACCAGTGACCGCCTTGCT CCTGCCGCTGGCCTT GCTGCTCCACGCCGCCAG
GCCGGACTTCGTGAT GACTCAGTCTCCTAGCTCCCT GACCGTGACAGCCGGCGAGAAGG
TGACCATGTCCTGCAAATCTAGTCAGAGTCT GCTGAACTCAGGCAATCAGAAGAACTAT
CTGACATGGTACCTGCAGAAGCCAGGGCAGCCCCCTAAACT GCTGATCTATTGGGCCAG
CACCAGGGAATCCGGCGTGCCCGACAGATTCACCGGCTCCGGGTCTGGAACAGATTTTA
CTCT GACCATTTCAAGCGTGCAGGCCGAGGACCTGGCTGTGTACTATTGT CAGAAT GAT
TACAGCTATCCCTACACATTTGGCGGGGGAACTAAGCTGGAAATCAAAGGTGGT GGTGG
TTCT GGTGGTGGT GGTT CCGGCGGCGGCGGCTCCGGTGGTGGTGGATCCGAGGT
GCAGC TGCAGCAGAGTGGACCCGAACT GGTGAAACCTGGCGCCTCCGTGAAAATGTCTT
GCAAG GCTAGTGGGTACACCTT CACAGACTACTATATGAAATGGGT
GAAGCAGTCACACGGGAA
GAGCCTGGAGTGGATCGGAGATATCATTCCCTCTAACGGCGCCACTTTCTACAATCAGA
AGTTTAAAGGCAAGGCTACTCT GACCGTGGACCGGAGCTCCTCTACCGCCTATATGCAC
CTGAACAGTCTGACATCAGAAGATAGCGCTGTGTACTATTGTACACGGTCCCAT CTGCT
GAGAGCCT CTTGGTTTGCTTATTGGGGCCAGGGGACACTGGTGACTGTGAGCTCCGCTA
GCACCACGACGCCAGCGCCGCGACCACCAACACCGGCGCCCACCATCGCGTCGCAGCCC
CTGT CCCT GCGCCCAGAGGCGT
GCCGGCCAGCGGCGGGGGGCGCAGTGCACACGAGGGG
GCTGGACTTCGCCTGTGATTTTTGGGTGCTGGTGGT GGTTGGTGGAGTCCTGGCTTGCT
ATAGCTTGCTAGTAACAGTGGCCTTTATTATTTTCT GGGTG

b. Illustrative Transmembrane Domains and Adapters

In some embodiments, the constructs described herein incorporate a transmembrane (TM) domain typically consisting of a hydrophobic a helix that spans the cell membrane. Illustrative TM domain include, for example, NKG2D, FcγRIIIa, NKp44, NKp30, NKp46, actKIR, NKG2C, CD8a, CD28 or IL15Rb. Illustrative TM adapters can include, for example, any endogenous TM adapter capable of signaling activation. For example, the TM adapter can be selected from FceRI y (ITAMxl), Oü3z (ITAMx3), DAP 12 (ITAMxl) or DAP 10 (YxxM/YINM).

c. Illustrative Hinges (Spacers)

The hinge, also referred to as a spacer, is in the extracellular structural region of the CAR that separates the binding units from the transmembrane domain. The hinge can be any moiety capable of ensuring proximity of the cell of interest to the target (e.g., NKG2-based hinge, TMa-based hinge, CD8-based hinge). With the exception of a few CARs based on the entire extracellular moiety of a receptor, such as NKG2D, as described herein, the majority of CAR (such as CAR T) cells are designed with immunoglobulin (Ig)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly. Illustrative hinge and TM sequences are provided below.

d. Hinge/Transmembrane (TM) Domain Sequences

NKG2D (Hinge(SEQ ID NO: 6)/TM(SEQ ID NO: 7))
TCCACAAGAATCAAGATCTTCCCTCTCTGAGCAGGAATCCTTTGTGCATTGAAGACTTT
AGATTCCTCTCTGCGGTAGACGTGCACTTATAAGTATTTGATGGGGTGGATTCGTGGTC
GGAGGTCTCGACACAGCTGGGAGATGAGTGAATTTCATAATTATAACTTGGATCTGAAG
AAGAGTGATTTTTCAACACGATGGCAAAAGCAAAGATGTCCAGTAGTCAAAAGCAAATG
TAGAGAAAATGCATCT/ CCATTTTTTTTCTGCTGCTTCATCGCTGTAGCCATGGGAATC
CGTTTCATTATTATGGTAACAATATGGAGT
FcyRIIIa (Hinge(SEQ ID NO: 8)/TM(SEQ ID NO: 9))
CACCTGAGGTGTCACAGCTGGAAGAACACTGCTCTGCATAAGGTCACATATTTACAGAA
TGGCAAAGGCAGGAAGTATTTTCATCATAATTCTGACTTCTACATTCCAAAAGCCACAC
TCAAAGACAGCGGCTCCTACTTCTGCAGGGGGCTTTTTGGGAGTAAAAATGTGTCTTCA
GAGACT GT GAACAT CAC CAT CACT CAAGGTT T GGCAGT GT C AACC AT CT C AT CATT
CTT TCCACCTGGGTACCAA/ GTCTCTTTCTGCTTGGTGATGGTACTCCTTTTTGCAGTGGAC
ACAGGACT AT ATT T CT CT GT GAAGAC AAACA
NKp44 (Hinge(SEQ ID NO: 10)/TM(SEQ ID NO: 11))
TGTAGAATCTACCGCCCTTCTGACAACTCTGTCTCTAAGTCCGTCAGATTCTATCTGGT
GGTATCTCCAGCCTCTGCCTCCACACAGACCTCCTGGACTCCCCGCGACCTGGTCTCTT
CACAGACCCAGACCCAGAGCTGTGTGCCTCC CACT GCAGGAGCCAGACAAGCCCCT GAG
TCTCCATCTACCATCCCTGTCCCTTCACAGCCACAGAACTCCACGCTCCGCCCTGGCCC
TGCAGCCCCCATTGCC/ CTGGTGCCTGTGTTCTGTGGACTCCTCGTAGCCAAGAGCCTG
GTGCTGTCAGCCCTGCTCGTCTGGTGGGGG
NKp30 (Hinge(SEQ ID NO: 12)/TM(SEQ ID NO: 13))
TCCGTCACGTGGTTCCGAGATGAGGTGGTTCCAGGGAAGGAGGTGAGGAATGGAACCCC
AGAGTTCAGGGGCCGCCTGGCCCCACTTGCTTCTTCCCGTTTCCTCCATGACCACCAGG
CTGAGCTGCACATCCGGGACGTGCGAGGCCATGACGCCAGCATCTACGTGTGCAGAGTG
GAGGTGCTGGGCCTTGGTGTCGGGACAGGGAATGGGACTCGGCTGGTGGTGGAGAAAGA
ACATCCTCAGCTAGGG/ GCTGGTACAGTCCTCCTCCTTCGGGCTGGATTCTATGCTGTC
AGCTTTCTCTCTGTGGCCGTGGGCAGCACC
NKp46 (Hinge(SEQ ID NO: 14)/TM(SEQ ID NO: 15))
TTCCCCCTGGGCCCTGTGACCACAGCCCACAGAGGGACATACCGATGTTTTGGCTCCTA
TAACAACCATGCCTGGTCTTTCCCCAGTGAGCCAGTGAAGCTCCTGGTCACAGGCGACA
TTGAGAACACCAGCCTTGCACCTGAAGACCCCACCTTTCCTGCAGACACTTGGGGCACC
TACCTTTTAACCACAGAGACGGGACTCCAGAAAGACCATGCCCTCTGGGATCACACTGC
CCAGAATCTCCTTCGG/ ATGGGCCTGGCCTTTCTAGTCCTGGTGGCTCTAGTGTGGTTC
CTGGTTGAAGACTGGCTCAGCAGGAAGAGG
Activating KIR (KIR2DS4)(Hinge(SEQ ID NO: 16)/TM(SEQ ID NO: 17))
AGGGAAGGGGAGGCCCATGAACGTAGGCTCCCTGCAGTGCGCAGCATCAACGGAACATT
CCAGGCCGACTTT CCTCTGGGCCCTGCCACCCACGGAGGGACCTACAGAT GCTT CGGCT
CTTT CCGT GACGCTCCCTACGAGTGGTCAAACTCGAGTGAT CCACTGCTT GTTT CCGTC
ACAGGAAACCCTT CAAATAGTT GGCCTTCACCCACT GAACCAAGCTCCAAAACCGGTAA
CCCCAGACACCTACAT / GTTCT GATT GGGACCTCAGTGGTCAAAATCCCTTTCACCATC
CTCCTCTT CTTTCTCCTTCATCGCTGG
NKG2C (Hinge(SEQ ID NO: 18)/TM(SEQ ID NO: 19))
AT GAATAAACAAAGAGGAACCTTCTCAGAAGTGAGT CTGGCCCAGGACCCAAAGCGGCA
GCAAAGGAAACCTAAAGGCAATAAAAGCTCCATTTCAGGAACCGAACAGGAAATATTCC
AAGTAGAATTAAATCTT CAAAATCCTTCCCT GAATCATCAAGGGATTGATAAAATATAT
GACT GCCAAGGTTTACT GCCACCTCCAGAGAAG/ CT CACTGCCGAGGTCCTAGGAATCA
TTTGCATT GTCCT GATGGCCACTGTGTTAAAAACAATAGTT CTTATTCCTTTC
CD8a (Hinge(SEQ ID NO: 20)/TM(SEQ ID NO: 21))
GTCCTCACCCTGAGCGACTTCCGCCGAGAGAACGAGGGCTACTATTTCTGCTCGGCCCT
GAGCAACT CCATCATGTACTTCAGCCACTTCGTGCCGGTCTTCCT GCCAGCGAAGCCCA
CCACGACGCCAGCGCCGCGACCACCAACACCGGCGCCCACCATCGCGTCGCAGCCCCTG
TCCCTGCGCCCAGAGGCGTGCCGGCCAGCGGCGGGGGGCGCAGTGCACACGAGGGGGCT
GGACTTCGCCTGT GAT /ATCTACATCTGGGCGCCCTTGGCCGGGACTTGT GGGGTCCTT
CTCCTGTCACTGGTTAT CACCCTTTACTGC
IL15Rb (Hinge(SEQ ID NO: 22)/TM(SEQ ID NO: 23))
GCCT CCCACTACTTTGAAAGACACCT GGAGTTCGAGGCCCGGACGCTGTCCCCAGGCCA
CACCTGGGAGGAGGCCCCCCTGCTGACTCTCAAGCAGAAGCAGGAATGGATCTGCCTGG
AGACGCTCACCCCAGACACCCAGTAT GAGTTTCAGGTGCGGGTCAAGCCT CTGCAAGGC
GAGTTCACGACCT GGAGCCCCT GGAGCCAGCCCCTGGCCTT CAGGACAAAGCCT GCAGC
CCTT GGGAAGGACACC /ATTCCGTGGCTCGGCCACCTCCTCGTGGGCCTCAGCGGGGCT
TTTGGCTT CATCATCTTAGTGTACTT GCTGATCAACTGCAGG

d. Illustrative Intracellular Signaling Domains (Costimulatory Domains)

In some embodiments, the CAR construct comprises one or more intracellular signaling domains. In some embodiments, the one or more intracellular signaling domains are active and effective in NK cells.

In some embodiments, an intracellular signaling domain can be any co-activating receptor capable of functioning in an NK cell (e.g., a ML NK cell). For example, a co-activating receptor can be CD137/41 BB (TRAF, NFkB), DNAM-1 (Y-motif), NKp80 (Y-motif), 2B4 (SLAMF) ITSM, CRACC (CS1/SLAMF7) ITSM, CD2 (Y-motifs, MAPK/Erk), CD27 (TRAF, NFkB), or integrins (e.g., multiple integrins).

In some embodiments, an intracellular signaling domain can be a cytokine receptor capable of functioning in an NK cell (e.g., a ML NK cell). For example, a cytokine receptor can be a cytokine receptor associated with persistence, survival, or metabolism, such as IL-2/15Rbyc::Jak1/3, STAT3/5, PI3K/mTOR, MAPK/ERK. As another example, a cytokine receptor can be a cytokine receptor associated with activation, such as IL-18R::NFkB. As another example, a cytokine receptor can be a cytokine receptor associated with IFN-g production, such as IL-12R STAT4. As another example, a cytokine receptor can be a cytokine receptor associated with cytotoxicity or persistence, such as IL-21 R Jak3/Tyk2, or STAT3.

In some embodiments, an intracellular signaling domain can be a TM adapter, such as FceRI y (ITAMxl), Oü3z (ITAMx3), DAP 12 (ITAMxl), or DAP (YxxM/YINM).

In some embodiments, CAR intracellular signaling domains (also known as endodomains) can be derived from costimulatory molecules from the CD28 family (such as CD28 and ICOS) or the tumor necrosis factor receptor (TNFR) family of genes (such as 4-1 BB, 0X40, or CD27). The TNFR family members signal through recruitment of TRAF proteins and are associated with cellular activation, differentiation, and survival.

In some embodiments, CD28 and 4-1 BB are used as costimulatory endodomains in CARs. In some embodiments, the high effector function and self-limited expansion of CD28-based CARs may be ideal to transiently treat diseases with a rapid tumor elimination and short-term persistence of the CAR in ML NK cells (i.e., as a bridge therapy for allogeneic hematopoietic stem cell transplantation). Furthermore, 4-1 BB-based CARs may be used to treat diseases in which complete response may require sustained NK cell persistence.

In some embodiments, other domains, such as incorporation of ICOS can be used.

In some embodiments, a CAR cell can join the properties of different intracellular domains by combining two or more intracellular domains in a CAR.

For example, such combinations can include one intracellular domain from the CD28 family and one intracellular domain from the TNFR family, resulting in the simultaneous activation of different signaling pathways.

In some embodiments, a illustrative costimulatory domains useful according to the present disclosure is a sequence such as those set out below.

CD137/41BB
(SEQ ID NO: 24)
aaacggggcagaaagaaactcctgtatatattcaaacaaccatttatgagaccagtacaaactactcaagaggaagat
ggctgtagctgccgatttccagaagaagaagaaggaggatgtgaactg
DNAM-1
(SEQ ID NO: 25)
aaggagaaggagagagagaagagatctatttacagagtcctgggatacacagaaggcacccaataactatagaagtc
ccatctctaccagtcaacctaccaatcaatccatggatgatacaagagaggatatttatgtcaactatccaaccttctctc
gcagaccaaagactagagt ttaag
NKp80
(SEQ ID NO: 26)
tttctcagggagtattgctaaaatgccaaaaaggaagttgttcaaatgccactcagtatgaggacactggagatctaaaa
gtgaataatggcacaagaagaaatataagtaataaggacctttgtgcttcgagatctgcagaccagacagtactatgcc
aatcagaatggctcaaataccaagggaagtgttattggttctctaatgagatgaaaagctggagtgacagttatgtgtat
tgtttggaaagaaaatctcatctactaatcatacatgaccaacttgaaatggcttttatacagaaaaacctaagacaatt
aaactacgtatggattgggcttaactttacctccttgaaaatgacatggacttgggtggatggttctccaatagattcaaa
gatattcttcataaagggaccagctaaagaaaacagctgtgctgccattaaggaaagcaaaattttctctgaaacctgca
gcagtgttttcaaatggatttgtcagtattag
2B4
(SEQ ID NO: 27)
agaccagtcccaaggaatttttgacaatttacgaagatgtcaaggatctgaaaaccaggagaaatcacgagcaggagc
agacttttcctggaggggggagcaccatctactctatgatccagtcccagtcttctgctcccacgtcacaagaacctgcat
atacattatattcattaattcagccttccaggaagtctggatccaggaagaggaaccacagcccttccttcaatagcacta
tctatgaagtg
NTBA
(SEQ ID NO: 28)
attccctatctttgtctactcagcgaacacagggccccgcagagtccgcaaggaacctagagtatgtttcagtgtctccaa
cgaacaacactgtgtatgcttcagtcactcattcaaacagggaaacagaaatctggacacctagagaaaatgatactat
cacaatttactccacaattaatcattccaaagagagtaaacccactttttccagggcaactgcccttgacaatgtc
gtgtaa
CRACC
(SEQ ID NO: 29)
agtacattgaagagaagaagagagtggacatttgtcgggaaactcctaacatatgcccccattctggagagaacacag
agtacgacacaatccctcacactaatagaacaatcctaaaggaagatccagcaaatacggtttactccactgtggaaat
accgaaaaag
CD2
(SEQ ID NO: 30)
aaaaggaaaaaacagaggagtcggagaaatgatgaggagctggagacaagagcccacagagtagctactgaagaa
aggggccggaagccccaccaaattccagcttcaacccctcagaatccagcaacttcccaacatcctcctccaccacctgg
tcatcgttcccaggcacctagtcatcgtcccccgcctcctggacaccgtgttcagcaccagcctcagaagaggcctcctgc
tccgtcgggcacacaagttcaccagcagaaaggcccgcccctccccagacctcgagttcagccaaaacctccccatggg
gcagcagaaaactcattgtccccttcctctaattaa
CD27
(SEQ ID NO: 31)
atccttgtgatcttctctggaatgttccttgttttcaccctggccggggccctgttcctccatcaacgaaggaaatatagatc
aaacaaaggagaaagtcctgtggagcctgcagagccttgtcgttacagctgccccagggaggaggagggcagcaccat
ccccatccaggaggattaccgaaaaccggagcctgcctgctccccctga
Integrins
A. ITGB1
(SEQ ID NO: 32)
aagcttttaatgataattcatgacagaagggagtttgctaaatttgaaaaggagaaaatgaatgccaaatgggacacgg
gtgaaaatcctatttataagagtgccgtaacaactgtggtcaatccgaagtatgagggaaaatga
B. ITGB2
(SEQ ID NO: 33)
aaggctctgatccacctgagcgacctccgggagtacaggcgctttgagaaggagaagctcaagtcccagtggaacaat
gataatccccttttcaagagcgccaccacgacggtcatgaaccccaagtttgctgagagttag
C. ITGB3
(SEQ ID NO: 34)
aaactcctcatcaccatccacgaccgaaaagaattcgctaaatttgaggaagaacgcgccagagcaaaatgggacaca
gccaacaacccactgtataaagaggccacgtctaccttcaccaatatcacgtaccggggcacttaa
IL15RB
(SEQ ID NO: 35)
aactgcaggaacaccgggccatggctgaagaaggtcctgaagtgtaacaccccagacccctcgaagttcttttcccagct
gagctcagagcatggaggagacgtccagaagtggctctcttcgcccttcccctcatcgtccttcagccctggcggcctggc
acctgagatctcgccactagaagtgctggagagggacaaggtgacgcagctgctcctgcagcaggacaaggtgcctga
gcccgcatccttaagcagcaaccactcgctgaccagctgcttcaccaaccagggttacttcttcttccacctcccggatgc
cttggagatagaggcctgccaggtgtactttacttacgacccctactcagaggaagaccctgatgagggtgtggccgggg
cacccacagggtc
ttccccccaacccctgcagcctctgtcaggggaggacgacgcctactgcaccttcccctccagggatgacctgctgctctt
ctcccccagtctcctcggtggccccagccccccaagcactgcccctgggggcagtggggccggtgaagagaggatgccc
ccttctttgcaagaaagagtccccagagactgggacccccagcccctggggcctcccaccccaggagtcccagacctgg
tggattttcagccaccccctgagctggtgctgcgagaggctggggaggaggtccctgacgctggccccagggagggagt
cagtttcccctggtccaggcctcctgggcagggggagttcagggcccttaatgctcgcctgcccctgaacactgatgccta
cttgtccctccaagaactccagggtcaggacccaactcacttggtgtag
IL18R
(SEQ ID NO: 36)
tataaagttgacttggttctgttctataggcgcatagcggaaagagacgagacactaacagatggtaaaacatatgatgc
ctttgtgtcttacctgaaagagtgtcatcctgagaataaagaagagtatacttttgctgtggagacgttacccagggtcct
ggagaaacagtttgggtataagttatgcatatttgaaagagatgtggtgcctggcggagctgttgtcgaggagatccatt
cactgatagagaaaagccggaggctaatcatcgttctcagccagagttacctgactaacggagccaggcgtgagctcga
gagtggactccacgaagcactggtagagaggaagattaagatcatcttaattgagtttactccagccagcaacatcacct
ttctccccccgtcgctgaaactcctgaagtcctacagagttctaaaatggagggctgacagtccctccatgaactcaaggt
tctggaagaatcttgtttacctgatgcccgcaaaagccgtcaagccatggagagaggagtcggaggcgcggtctgttctc
tcagcaccttga
IL12R
IL12RB1
(SEQ ID NO: 37)
aacagggccgcacggcacctgtgcccgccgctgcccacaccctgtgccagctccgccattgagttccctggagggaagg
agacttggcagtggatcaacccagtggacttccaggaagaggcatccctgcaggaggccctggtggtagagatgtcctg
ggacaaaggcgagaggactgagcctctcgagaagacagagctacctgagggtgcccctgagctggccctggatacaga
gttgtccttggaggatggagacaggtgcaaggccaagatgtga
IL12RB2
(SEQ ID NO: 38)
cattacttccagcaaaaggtgtttgttctcctagcagccctcagacctcagtggtgtagcagagaaattccagatccagca
aatagcacttgcgctaagaaatatcccattgcagaggagaagacacagctgcccttggacaggctcctgatagactggc
ccacgcctgaagatcctgaaccgctggtcatcagtgaagtccttcatcaagtgaccccagttttcagacatcccccctgct
ccaactggccacaaagggaaaaaggaatccaaggtcatcaggcctctgagaaagacatgatgcacagtgcctcaagcc
caccacctccaagagctctccaagctgagagcagacaactggtggatctgtacaaggtgctggagagcaggggctccg
acccaaagcccgaaaacccagcctgtccctggacggtgctcccagcaggtgaccttcccacccatgatggctacttaccc
tccaacatagatgacctcccctcacatgaggcacctctcgctgactctctggaagaactggagcctcagcacatctccctt
tctgttttcccctcaagttctcttcacccactcaccttctcctgtggtgataagctgactctggatcagttaaagatgaggtg
tgactccctcatgctctga
IL21R
(SEQ ID NO: 39)
agcctgaagacccatccattgtggaggctatggaagaagatatgggccgtccccagccctgagcggttcttcatgcccct
gtacaagggctgcagcggagacttcaagaaatgggtgggtgcacccttcactggctccagcctggagctgggaccctgg
agcccagaggtgccctccaccctggaggtgtacagctgccacccaccacggagcccggccaagaggctgcagctcacg
gagctacaagaaccagcagagctggtggagtctgacggtgtgcccaagcccagcttctggccgacagcccagaactcg
gggggctcagcttacagtgaggagagggatcggccatacggcctggtgtccattgacacagtgactgtgctagatgcag
aggggccatgcacctggccctgcagctgtgaggatgacggctacccagccctggacctggatgctggcctggagcccag
cccaggcctagaggacccactcttggatgcagggaccacagtcctgtcctgtggctgtgtctcagctggcagccctgggc
taggagggcccctgggaagcctcctggacagactaaagccaccccttgcagatggggaggactgggctgggggactgc
cctggggtggccggtcacctggaggggtctcagagagtgaggcgggctcacccctggccggcctggatatggacacgtt
tgacagtggctttgtgggctctgactgcagcagccctgtggagtgtgacttcaccagccccggggacgaaggaccccccc
ggagctacctccgccagtgggtggtcattcctccgccactttcgagccctggaccccaggccagctaa
IRE1a
(SEQ ID NO: 40)
cccctgagcatgcatcagcagcagcagctccagcaccagcagttccagaaggaactgga
gaagatccagctcctgcagcagcagcagcagcagctgcccttccacccacctggagacacggctcaggacggcgagct
cctggacacgtctggcccgtactcagagagctcgggcaccagcagccccagcacgtcccccagggcctccaaccactcg
ctctgctccggcagctctgcctccaaggctggcagcagcccctccctggaacaagacgatggagatgaggaaaccagcg
tggtgatagttgggaaaatttccttctgtcccaaggatgtcctgggccatggagctgagggcacaattgtgtaccggggc
atgtttgacaaccgcgacgtggccgtgaagaggatcctccccgagtgttttagcttcgcagaccgtgaggtccagctgttg
cgagaatcggatgagc
acccgaacgtgatccgctacttctgcacggagaaggaccggcaattccagtacattgccatcgagctgtgtgcagccacc
ctgcaagagtatgtggagcagaaggactttgcgcatctcggcctggagcccatcaccttgctgcagcagaccacctcgg
gcctggcccacctccactccctcaacatcgttcacagagacctaaagccacacaacatcctcatatccatgcccaatgca
cacggcaagatcaaggccatgatctccgactttggcctctgcaagaagctggcagtgggcagacacagtttcagccgcc
gatctggggtgcctggcacagaaggctggatcgctccagagatgctgagcgaagactgtaaggagaaccctacctaca
cggtggacatcttttctgcaggctgcgtcttttactacgtaatctctgagggcagccacccttttggcaagtccctgcagcg
gcaggccaacatcctcctgggtgcctgcagccttgactgcttgcacccagagaagcacgaagacgtcattgcacgtgaat
tgatagagaagatgattgcgatggatcctcagaaacgcccctcagcgaagcatgtgctcaaacacccgttcttctggagc
ctagagaagcagctccagttcttccaggacgtgagcgacagaatagaaaaggaatccctggatggcccgatcgtgaag
cagttagagagaggcgggagagccgtggtgaagatggactggcgggagaacatcactgtccccctccagacagacctg
cgtaaattcaggacctataaaggtggttctgtcagagatctcctccgagccatgagaaataagaagcaccactaccggg
agctgcctgcagaggtgcgggagacgctggggtccctccccgacgacttcgtgtgctacttcacatctcgcttcccccacc
tcctcgcacacacctaccgggccatggagctgtgcagccacgagagactcttccagccctactacttccacgagccccca
gagccccagcccccagtgactccagacgccctctga

Optionally, an extracellular signaling domain can be incorporated into the CAR construct to propagate signaling. The extracellular signaling domain can be cloned into the hinge region, such as a CD8 hinge, but can be chosen based on the target.

### Methods of Preparation and Use

The present disclosure also relates generally to methods for the preparation and use of the vectors and cells described herein. Methods for the manipulation, propagation, transformation, etc., of immune effector cells is well known and established. Essentially any known and available techniques can be used to carry out the present disclosure accordingly.

In certain particular embodiments, the present disclosure provides a method of generating chimeric antigen receptor NK (CAR NK) cells or chimeric antigen receptor memory-like NK (CARML NK) cells. The isolated NK cells can be activated using cytokines, such as IL-12/15/18. The NK cells can be incubated in the presence of the cytokines for an amount of time sufficient to form cytokine-activated memory-like (ML) NK cells. For example, the amount of time sufficient to form cytokine-activated memory-like (ML) NK cells can be between about 8 and about 24 hours, about 12 hours, or about 16 hours. As another example, the amount of time sufficient to form cytokine-activated memory-like (ML) NK cells can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Next, the chimeric antigen receptor (CAR) can be transduced via a viral vector (e.g., lentivirus) into the cytokine-activated ML NK cells in the presence of cytokines for an amount of time sufficient to virally transduce CAR into the cytokine-activated ML NK cells, resulting in CAR-transduced ML NK cells. For example, the amount of time sufficient to form CAR-transduced ML NK cells can be between about 12 hours and about 24 hours. As another example, the amount of time sufficient to virally transduce CAR into the ML NK cells (forming CAR-transduced ML NK cells) can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Alternatively, the CAR can be delivered to the cytokine-activated NK cell using a non-viral vector delivery system, such as a transposon.

Next, the CAR-transduced ML NK cells can be incubated in the presence of IL-15 for an amount of time sufficient to express the vector and to form CAR-expressing ML NK (CARML NK cells). For example, the amount of time sufficient to form CARML NK cells can be between about 3 days and about 8 days. As an example, the amount of time sufficient to form CARML NK cells can be at least about 1 day; about 2 days; about 3 days; about 4 days; about 5 days; about 6 days; about 7 days; about 8 days; about 9 days; about 10 days; about 11 days; about 12 days; about 13 days; or about 14 days.

In other embodiments, methods for preparing ML NK cells to be used according to the present disclosure include those described in WO2020/047299 and WO2020/047473, the contents of which are incorporated herein by reference in their entireties.

### Formulations

The components and compositions (e.g., sequences, vectors, cells) described herein can be formulated by any conventional manner using one or more pharmaceutically acceptable carriers or excipients as described in, for example, Remington's Pharmaceutical Sciences (A. R. Gennaro, Ed.), 21 st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21 st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. To maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided according to the present disclosure are methods of using the genetically modified cells described herein for stimulating an immune response or for treating disease or disorder in a subject in need thereof.

In some embodiments, the method can be a method of treating a proliferative disease, disorder, or condition, infectious disease, or immune disorder in a subject in need of administration of a therapeutically effective amount of cell-based therapy (e.g., using genetically modified immune cells). The disclosed cell-based therapy can be used as a treatment for cancer (e.g., as an immunotherapy drug), for an autoimmune disease (e.g., treatment to deplete B cells), or for an infectious disease.

In some more particular embodiments, the methods employ genetically modified NK cells or ML NK cells as described herein.

The scFvs described herein can be used for hematological malignancies such as AML, ALL, or Lymphoma, but can also be expanded for use in any malignancy, autoimmune, or infectious disease where a scFv can be generated against a target. For example, the constructs described herein can be used to treat or prevent autoimmunity associated with auto-antibodies (similar indications as rituximab for autoimmunity). As another example, the disclosed constructs can also be applied to virally infected cells, using a scFv that can recognize viral antigens, for example gp120 and gp41 on HIV-infected cells.

Methods described herein are generally performed on a subject in need thereof. A subject in need of the therapeutic methods described herein can be a subject having, diagnosed with, suspected of having, or at risk for developing a proliferative disease, disorder, or condition; an immune disorder; or an infectious disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a cell-based treatment is, for example, that amount that would cause the desired therapeutic effect in a subject while minimizing undesired side effects. In various embodiments, an effective amount of a NK cell-based treatment described herein can substantially inhibit a disease, disorder, or condition, slow the progress of a disease, disorder, or condition, or limit the development of a disease, disorder, or condition.

Substantially can be any large portion up to totality. Thus “substantially blocked or inhibited,” or “substantially removed” can be nearly or nearly completely blocked, inhibited, or removed.

According to the methods described herein, administration can be parenteral, pulmonary, oral, topical, intradermal, intramuscular, intraperitoneal, intravenous, subcutaneous, intranasal, epidural, ophthalmic, buccal, or rectal administration. Preferably, genetically modified cells can be administered as an intravenous infusion.

When used in the treatments described herein, a therapeutically effective amount of a NK cell-based treatment can be employed in a purified form or, where such forms exist, in pharmaceutically acceptable form and with or without a pharmaceutically acceptable excipient. For example, the compounds of the present disclosure can be administered, at a reasonable benefit/risk ratio applicable to any medical treatment, in a sufficient amount to inhibit a disease, disorder, or condition, slow the progress of a disease, disorder, or condition, or limit the development of a disease, disorder, or condition.

The amount of cell-based treatment (e.g., CAR NK cells or CARML NK cells) described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the Ldso (the dose lethal to 50% of the population) and the Edso, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the cell-based treatment can occur as a single event or over a time course of treatment. For example, cell-based treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more. Treatment in accord with the methods described herein can be performed prior to, concurrent with, or after conventional treatment modalities for a disease, disorder, or condition, such as chemotherapy, immunotherapy, or checkpoint blockade therapy. For example, a subject can be administered at least one therapeutic agent selected from an interferon; a checkpoint inhibitor antibody; an antibody-drug conjugate (ADC); an anti-HLA-DR antibody; or an anti-CD74 antibody. Other examples can include a therapeutic agent selected from a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound, or a radioisotope.

In some embodiments, a cell-based treatment can be administered simultaneously or sequentially with another agent, such as an antibiotic, an anti-inflammatory, or another agent. For example, a cell-based treatment can be administered simultaneously with another agent, such as an antibiotic or an anti-inflammatory. Simultaneous administration can occur through administration of separate compositions, each containing one or more of a cell-based treatment, an antibiotic, an anti-inflammatory, or another agent. Simultaneous administration can occur through administration of one composition containing two or more of a cell-based treatment, an antibiotic, an anti-inflammatory, or another agent. A cell-based treatment can be administered sequentially with an antibiotic, an anti-inflammatory, or another agent. For example, a cell-based treatment can be administered before or after administration of an antibiotic, an anti-inflammatory, or another agent.

In some embodiments, methods and compositions as described herein can be used for the prevention, treatment, or slowing the progression of cancer, autoimmune conditions associated with autoantibodies, immune disorder, or infectious diseases (e.g., bacterial, viral). The disclosed CARML NK cell constructs can be designed to incorporate a targeting antibody fragment against a disease-associated antigen, such as scFvs that target cancer or an infectious disease. As described herein, targeting antibody fragments against a disease-associated antigen are well known.

For example, the cancer can a hematological cancer or a cancer with a solid tumor. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

In some embodiments, the autoimmune condition or immune disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Balo disease; Behcet's disease; Benign mucosal pemphigoid; Bullous pemphigoid; Castleman disease (CD); Celiac disease; Chagas disease; Chronic inflammatory demyelinating polyneuropathy (CI DP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressier s syndrome; Endometriosis; Eosinophilic esophagitis (EoE); Eosinophilic fasciitis; Erythema nodosum, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammaglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB, Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, Ill; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PROA); Pyoderma gangrenosum, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjogren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC), Undifferentiated connective tissue disease (UCTD), Uveitis; Vasculitis; Vitiligo; or Vogt-Koyanagi-Harada Disease.

In some embodiments, the autoimmune condition or immune disorder can be an autoinflammatory disease. The autoinflammatory can be Familial Mediterranean Fever (FMF), neonatal Onset Multisystem Inflammatory Disease (NOMID), Tumor Necrosis Factor Receptor-Associated Periodic Syndrome (TRAPS), Deficiency of the Interleukin-1 Receptor Antagonist (DIRA), Behget's Disease, or Chronic Atypical Neutrophilic Dermatosis with Lipodystrophy and Elevated Temperature (CANDLE).

In some embodiments, the treatment of an infectious disease can be any bacterial infection or viral infection, using a scFv that can recognize antigens, such as antigens on HIV infected cells. The infectious disease can be Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia, Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1, 2, 3, 4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin-B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock jaw); Trichomoniasis (Trichomonas infection); Trichonosis Infection (Trichinosis); Tuberculosis (TB); Tuberculosis (Latent) (LTBI); Tularemia (Rabbit fever); Typhoid Fever, Group D, Typhus; Vaginosis, bacterial (Yeast Infection); Vaping-Associated Lung Injury (e-Cigarette Associated Lung Injury); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

### Administration

In some embodiments, the genetically modified cells as described herein (e.g., CARML NK cells, modified NK cells, pre-activated NK cells, NKG2A-blocked NK cells, pre-activated and NKG2A-blocked NK cells) are directly administered to a subject.

In embodiments using NK cells, the NK cells can be purified and activated with IL-12/IL-15/IL-18 using known methods. The NK cells are transduced, for example with a viral vector (e.g., CAR lentiviral, adenoviral, NNV vector, etc.) or electroporated with CAR transposon. The cells can be infused into the patient typically at a dose of between about 1e6 and about 1e8 cells/kg. In the haplo/allo setting the cells can be supported with rhIL-2 and in the autologous setting the cells can be supported with IL-15.

Administration can be by any suitable route, e.g., parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 pm), nanospheres (e.g., less than 1 pm), microspheres (e.g., 1-100 pm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner like that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent In vivo\ prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

The practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience, Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1: An MND-HTLV Hybrid Promoter Drives Superior Expression in NK Cells

A promoter referred to as MND has been previously described as a myeloproliferative sarcoma virus enhancer, negative control region (NCR) deleted, d1587rev primer-binding site (PBS) substituted promoter (Challita et al., J Virol 69(2):748-55 (1995); Robbins et al., J Virol 71(12):9466-74 (1997). MND has been shown to be able to drive expression of heterologous sequences in different cell types, including some immune cell types (Ho et al., Mol Ther Methods Clin Dev Mar 13; 21:237-246 (2021).

In an effort to identify improved promoters capable of driving robust and sustained expression of heterologous sequences in immune cells, particularly NK cells, regulatory sequences derived from MND and Human T-Lymphotropic Virus (HTLV) were fused to generate the non-naturally occurring hybrid sequences referred to as MND-HTLV-1 (SEQ ID NO: 1) and MND-HTLV-2 (SEQ ID NO: 2). Additional details about these hybrid promoter sequences are shown in FIGS. 1 and 2.

Expression studies were carried out using a lentiviral-based GFP system to test the expression levels driven by the MND-HTLV promoter in NK cells against a number of other promoters. Briefly, NK were isolated from a frozen leukopak with a Miltenyi MultiMACS protocol. Cells were treated overnight with “WU-PRIME” (a chimeric fusion protein comprising portions of IL-12, IL-15 and IL-18) before treatment for two days with “WU-EXPAND” (a chimeric fusion protein comprising portions of IL-7, IL-15 and IL-21). See, e.g., WO2020/047299 and WO2020/047473, respectively, the contents of which are incorporated herein by reference.

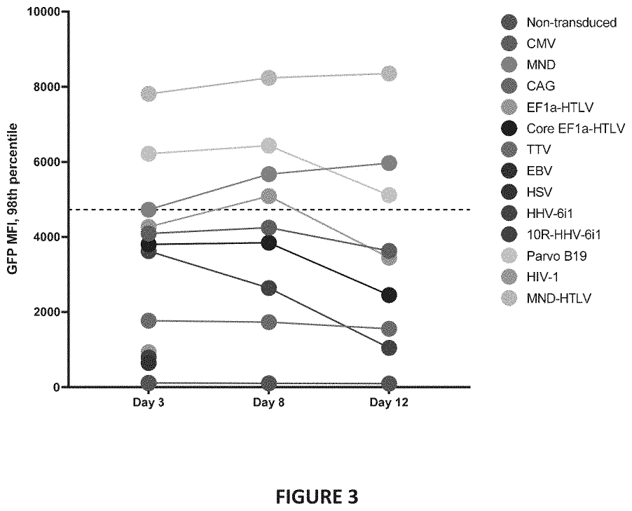

A lentiviral transduction was then performed. After washing the virus, the NK were allowed to proliferate with additional WU-EXPAND treatments taking place every other day. Transduced cells were then subjected to flow cytometry and GFP expression was measured within singlet, live, CD56+, CD3-NK cells at several time points during proliferation. Based on these experiments, as shown in FIG. 3, the MND-HTLV promoter demonstrated superior expression in NK cells to all other promoters tested and maintained high levels of expression even at 12 days post-transduction.

### Example 2. An MND-HTLV Hybrid Promoter Drives Superior Expression of a Cell Surface Receptor in NK Cells and Results in Superior Tumor Cell Killing

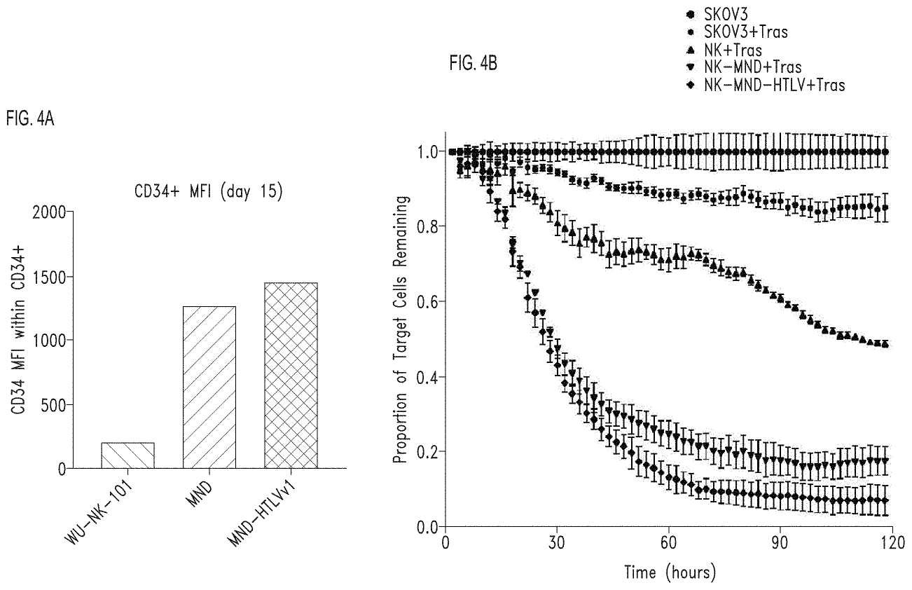

Using the MND-HTLV promoter identified in Example 1, expression and function studies were carried out using a receptor designed to enhance the antibody dependent cellular cytotoxicity of NK cells, compared to the MND promoter. The receptor is composed of a the promoter (MND or MND-HTLV), the CD16 extracellular domain, the CD16 transmembrane domain that is mutated (S197P) to prevent ADAMTS13 cleavage, and the 2B4, CD79A, CD79B and CD132 intracellular domains. Additionally, a CD34 extracellular tag is encoded after a P2A cleavage site. NK cells were isolated, transduced and expanded as in Example 1. The receptor expression was measured via using flow cytometry with an antibody staining for the CD34 tag. FIG. 4A shows that the MND-HTLV1 promoter led to higher receptor expression (MFI) compared to the MND promoter alone.

Next, the receptor expression NK cells were tested in a cytotoxicity assay against SKOV3 tumor cells in combination with the anti-HER2 ADCC antibody, trastuzumab. For cytotoxicity assays Fluorescently labeled SKOV3 tumor cells were plated in 96 well plates at 4,000 cells/well and incubated overnight at 37 degrees C. The next day, receptor expressing NK cells were added to wells at effector to target (E:T) ratios of 3:1, 1:1 and 1:3. Trastuzumab or isotype control antibody was added to wells with a final concentration of 3 ug/ml. Plates were placed in an Incucyte at 37 degrees C. and monitored for tumor cell fluorescence by scanning every 2 hours for 72 hours. FIG. 4B shows the results of the E:T of 1:1 cultures for five different conditions.
(1) target only—SKOV3 cells alone,
(2) target+tras—SKOV3 cells with Traztuzumab,
(3) NK+tras—SKOV3 cells co-cultured with naïve, NK cells in the presence of Traztuzumab,
(4) NK-MND+tras—SKOV3 cells co-cultured with NK cells transduced with MND promoter in the presence of Traztuzumab, and
(5) NK-MND-HTLV1—SKOV3 cells co-cultured with NK cells transduced MND-HLTV promoter, in the presence of Traztuzumab.

The data demonstrates that the NK cells that were transduced with the MND-HTLV1 promoter driven receptor led to increased antibody dependent cellular cytotoxicity of tumor in the presence of trastuzumab (FIG. 4B).

1. A nucleic acid vector comprising a hybrid promoter sequence operably linked to a heterologous sequence, where the hybrid promoter sequence has activity in immune cells, and where the hybrid promoter sequence comprises: (i) an MND promoter sequence and (ii) an HTLV enhancer sequence.

2. The vector of claim 1, where the hybrid promoter sequence comprises the sequence of SEQ ID NO: 1 or SEQ ID NO: 2 or a fragment or variant thereof having at least 90% identity thereto.

3. The vector of claim 1, where the MND promoter sequence comprises residues 1-357 of SEQ ID NO: 1 or residues 1-548 of SEQ ID NO: 2.

4. The vector of claim 1, where the HTLV enhancer sequence comprises residues 441-709 of SEQ ID NO: 1 or residues 549-817 of SEQ ID NO: 2.

5. The vector of claim 1, where the immune cell is selected from the group consisting of a T cell, a B cell, a macrophage, an NK-T cell and an NK cell.

6. The vector of claim 5, where the immune cell is an NK cell or ML NK cell.

7. The vector of claim 1, where the heterologous sequence encodes a chimeric antigen receptor (CAR).

8. The vector of claim 7, where the CAR comprises an extracellular domain that binds a target antigen, a transmembrane domain and one or more intracellular signaling domains.

9. The vector of claim 8, where the target antigen is selected from the group consisting of mesothelin, CD2, CD3, CD4, CD5, CD7, BAFF-R, gp120, gp41, BCMA, CD123, CD138, CD19, CD20, CD22, CD33, CD38, CD5, IgK, LeY, NKG2D-Ligands, MICA, MICB, ULBP1, ULBP2, ULBP3, ULBP4, ULBP5, ULBP6, ROR1, WT1, c-MET, CAIX, CD133, CD171, CD70, CEA, EGFR, EGFRvIII, EPCAM, EphA2, FAP, GD2, GPC3, Her2, HPV16-E6, IL13Ra2, LeY, MAGEA3, MAGEA4, MART1, MSLN, MUC1, MUC16, NY-ESO-1, PD-L1, PSCA, PSMA, ROR1, VEGFR2, BAFF-R and SLAM-F7.

10. The vector of claim 8, where the transmembrane domain is selected from the group consisting of NKG2D, FcγRIIIa, NKp44, NKp30, NKp46, actKIR, NKG2C, CD8a, CD28 and IL15Rb.

11. The vector of claim 8, where the intracellular signaling domain is selected from the group consisting of CD137/4-1BB, DNAM-1, NKp80, 2B4, NTBA, CRACC, CD2, CD27, CD79a CD79b, CD132, one or more integrins, IL-15R, IL-18R, IL-12R, IL-21 R, IRE1a, and combinations thereof.

12. The vector of claim 1, where the heterologous sequence encodes a cytokine, a chemokine, an antibody, an enzyme, an anti-tumor molecule or other therapeutic molecule.

13. The vector of claim 1, where the vector is a transposon.

14. The vector of claim 1, where the vector is a viral vector.

15. The vector of claim 14, where the viral vector is a lentiviral vector.

16. A genetically modified immune cell comprising a vector according to claim 1.

17. The genetically modified immune cell of claim 16, where the immune cell is an NK cell or ML NK cell.

18. The genetically modified immune cell of claim 16, where the cell has been modified to be deficient for NKG2A, CD8, EP2, EP4 and/or CISH expression, activity or signaling.

19. A genetically modified ML NK cell comprising a chimeric antigen receptor where the chimeric antigen receptor is expressed under the control of a hybrid promoter sequence comprising: (i) an MND promoter sequence and (ii) an HTLV enhancer sequence, wherein the cell is optionally deficient for NKG2A and/or CD8 expression, activity or signaling.

20. The genetically modified ML NK cell of claim 19, where the hybrid promoter sequence comprises the sequence of SEQ ID NO: 1 or SEQ ID NO: 2 or a fragment or variant thereof having at least 90% identity thereto.

21. The genetically modified ML NK cell of claim 19, where the MND promoter sequence comprises residues 1-357 of SEQ ID NO: 1 or residues 1-548 of SEQ ID NO: 2.

22. The genetically modified ML NK cell of claim 19, where the HTLV enhancer sequence comprises residues 441-709 of SEQ ID NO: 1 or residues 549-817 of SEQ ID NO: 2.

23. A method of inducing an immune response to a disease in a subject in need thereof comprising administering to the subject a genetically modified immune cell according to claim 16.

24. A method of treating cancer in a subject in need thereof comprising administering to the subject a genetically modified immune cell according to claim 16, wherein the target antigen is a cancer-associated target antigen.
